(19) United States
(12) Patent Application Publication
(10) Pub. No.: US 2019/0100799 A1
(43) Pub. Date: Apr. 4, 2019

(54) COMPOSITIONS AND METHODS OF DETECTING MYELITIS

(71) Applicant: Michael Levy, Phoenix, MD (US)

(72) Inventor: Michael Levy, Phoenix, MD (US)

(21) Appl. No.: 15/963,879

(22) Filed: Apr. 26, 2018

Related U.S. Application Data
(60) Provisional application No. 62/490,583, filed on Apr. 26, 2017.

(51) Int. Cl. C12Q 1/6876; C12Q 1/6853; C07H 21/04

(52) U.S. Cl. CPC C12Q 1/6876; C12Q 1/6853; C12Q 2600/118; C12Q 2600/158; C12Q 2600/112; C07H 21/04

(57) ABSTRACT

The present invention relates to novel compositions and methods for detecting myelitis. The invention also relates to methods of identifying mutations in VPS37a indicative of monophasic acute transverse myelitis.

### CROSS REFERENCE TO RELATED APPLICATIONS

This Application claims the benefit of priority of U.S. Provisional Application 62/490,583 filed on Apr. 26, 2017, the entire contents of which are incorporated herein by reference in their entirety.

### STATEMENT AS TO FEDERALLY SPONSORED RESEARCH

The invention was made with government support under the following grants awarded by the National Institutes of Health: NS078555 and CA163705. The government has certain rights in the invention.

### FIELD OF THE INVENTION

Embodiments of the invention are directed to compositions for detecting myelitis and their method of use.

### BACKGROUND

Myelitis involves the infection or the inflammation of the white matter or gray matter of the spinal cord which is a part of the central nervous system that acts as a bridge between the brain and the rest of the body. During an inflammatory response in the spinal cord, the myelin of the axon may be damaged causing symptoms such as paralysis and sensory loss. In particular, idiopathic transverse myelitis is a sub-type of myelitis that results from an acute inflammatory attack of the spinal cord, which leads to weakness, sensory loss, and bowel/bladder dysfunction. The prevalence is 0.1-0.2/100,000 and there are no known risk factors. Accordingly, there is an urgent need for compositions and methods for diagnosing myelitis.

### SUMMARY

The present disclosure provides compositions and methods for detecting myelitis. In particular, the present disclosure provides methods of identifying mutations in VPS37a indicative of monophasic acute transverse myelitis.

In one aspect, the present invention provides for a method of diagnosing myelitis, comprising: obtaining a sample from a subject; amplifying a region of a VPS37a gene with a first primer and a second primer, wherein the first primer has the sequence aaggcagtgtgagatgtgaaga (SEQ ID NO: 1) fragments or variants thereof, and the second primer has the sequence tcccactaaggcaacaacaa (SEQ ID NO: 2) fragments or variants thereof; sequencing the amplified sequence; and identifying a leucine to isoleucine change at amino acid position 234. In an embodiment, the subject is human. In an embodiment, the primer is coupled to a fluorescent probe.

In certain embodiments, an isolated nucleic acid sequence comprises SEQ ID NO: 1, SEQ ID NO: 2, fragments or variants thereof. In some embodiments, SEQ ID NO: 1 and/or SEQ ID NO: 2 are conjugated to a fluorescent probe or detectable label.

In certain embodiments, a composition comprises a nucleic acid sequence comprising SEQ ID NOS: 1, 2, fragments or variants thereof. In certain embodiments, the nucleic acid sequences are conjugated to a detectable label.

### Definitions

By “agent” is meant any small compound, antibody, nucleic acid molecule, or polypeptide, or fragments thereof.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

By “ameliorate” is meant decrease, suppress, attenuate, diminish, arrest, or stabilize the development or progression of a disease.

In this disclosure, “comprises,” “comprising,” “containing,” and “having” and the like may have the meaning ascribed to them in U.S. Patent law and may mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein, the terms “associated with,” “conjugated,” “linked,” “attached,” “coupled” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions.

“Detect” refers to identifying the presence, absence or amount of the agent (e.g., a nucleic acid molecule, for example deoxyribonucleic acid (DNA) or ribonucleic acid (RNA)) to be detected.

By “detectable label” is meant a composition that when linked (e.g., joined—directly or indirectly) to a molecule of interest renders the latter detectable, via, for example, spectroscopic, photochemical, biochemical, immunochemical, or chemical means. Direct labeling can occur through bonds or interactions that link the label to the molecule, and indirect labeling can occur through the use of a linker or bridging moiety which is either directly or indirectly labeled. Bridging moieties may amplify a detectable signal. For example, useful labels may include radioactive isotopes, magnetic beads, metallic beads, colloidal particles, fluorescent labeling compounds, electron-dense reagents, enzymes (for example, as commonly used in an enzyme-linked immunosorbent assay (ELISA)), biotin, digoxigenin, or haptens. When the fluorescently labeled molecule is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, p-phthaldehyde and fluorescamine. The molecule can also be detectably labeled using fluorescence emitting metals. These metals can be attached to the molecule using such metal chelating groups as diethylenetriaminepentacetic acid (DTPA) or ethylenediaminetetraacetic acid (EDTA). The molecule also can be detectably labeled by coupling it to a chemiluminescent compound. The presence of the chemiluminescent-tagged molecule is then determined by detecting the presence of luminescence that arises during the course of chemical reaction. Examples of particularly useful chemiluminescent labeling compounds are luminol, isoluminol, theromatic acridinium ester, imidazole, acridinium salt and oxalate ester.

A “detection step” may use any of a variety of known methods to detect the presence of nucleic acid.

By “diagnostic” is meant any method that identifies the presence of a pathologic condition or agent or characterizes the nature of a pathologic condition (e.g., an infection). Diagnostic methods differ in their sensitivity and specificity. While a particular diagnostic method may not provide a definitive diagnosis of a condition, it suffices if the method provides a positive indication that aids in diagnosis.

The term “epigenetic marker” or “epigenetic change” as used herein is meant to refer to a change in the DNA sequences or gene expression by a process or processes that do not change the DNA coding sequence itself (e.g., methylation, etc.). In an exemplary embodiment, methylation is an epigenetic marker.

The term “expression profile” is used broadly to include a genomic expression profile. Profiles may be generated by any convenient means for determining a level of a nucleic acid sequence, e.g., quantitative hybridization of microRNA, labeled microRNA, amplified microRNA, complementary/synthetic DNA (cDNA), etc., quantitative polymerase chain reaction (PCR), and ELISA for quantitation, and allow the analysis of differential gene expression between two samples. A subject or patient sample is assayed. Samples are collected by any convenient method, as known in the art. According to some embodiments, the term “expression profile” means measuring the relative abundance of the nucleic acid sequences in the measured samples.

By “fluorescent detection” is meant the measurement of the signal of a labeled moiety of at least one of the one or more nucleotides or nucleotide analogs. Sequencing using fluorescent nucleotides typically involves photobleaching the fluorescent label after detecting an added nucleotide. In some embodiments, fluorescent detection can include bead-based fluorescent, FRET, infrared labels, pyrophosphatase, ligase methods including labeled nucleotides or polymerase or use of cyclic reversible terminators. In some embodiments, fluorescent detection can include direct methods of nanopores or optical waveguide including immobilized single molecules or in solution. Photobleaching methods include a reduced signal intensity, which builds with each addition of a fluorescently labeled nucleotide to the primer strand. By reducing the signal intensity, longer DNA templates are optionally sequenced.

By “fragment” is meant a portion of a nucleic acid molecule. This portion contains, preferably, at least 10%, 20%, 30%, 40%, 50%, 60%, 70%, 80%, or 90% of the entire length of the reference nucleic acid molecule. For example, a fragment may contain 10, 20, 30, 40, 50, 60, 70, 80, 90, or 100, 200, 300, 400, 500, 600, 700, 800, 900, or 1000 nucleotides. However, the invention also comprises nucleic acid fragments, so long as they exhibit the desired biological activity of the full length nucleic acid, respectively. A nucleic acid fragment of almost any length is employed. For example, illustrative polynucleotide segments with total lengths of about 10,000, about 5000, about 3000, about 2,000, about 1,000, about 500, about 200, about 100, about 50 base pairs in length (including all intermediate lengths) are included in many implementations of this invention.

By “gene” is meant a locus (or region) of DNA that encodes a functional RNA or protein product (e.g., VPS37a), including upstream and downstream regulatory sequences, and is the molecular unit of heredity.

By “isolated nucleic acid” is meant a nucleic acid that is free of the genes which flank it in the naturally-occurring genome of the organism from which the nucleic acid is derived. The term covers, for example: (a) a DNA which is part of a naturally occurring genomic DNA molecule, but is not flanked by both of the nucleic acid sequences that flank that part of the molecule in the genome of the organism in which it naturally occurs; (b) a nucleic acid incorporated into a vector or into the genomic DNA of a prokaryote or eukaryote in a manner, such that the resulting molecule is not identical to any naturally occurring vector or genomic DNA; (c) a separate molecule such as a synthetic cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Isolated nucleic acid molecules according to the present invention further include molecules produced synthetically, as well as any nucleic acids that have been altered chemically and/or that have modified backbones. For example, the isolated nucleic acid is a purified eDNA or RNA polynucleotide. Isolated nucleic acid molecules also include messenger ribonucleic acid (mRNA) molecules.

The term “promoter” or “promoter region” refers to a minimal sequence sufficient to direct transcription or to render promoter-dependent gene expression that is controllable for cell-type specific or tissue-specific gene expression, or is inducible by external signals or agents. Promoters may be located in the 5′ or 3′ regions of the gene. Promoter regions, in whole or in part, of a number of nucleic acids can be examined for sites of variation and/or mutation. In general, a promoter includes, at least, 50, 75, 100, 125, 150, 175, 200, 250, 300, 400, 500, 750, 1000, 1500, or 2000 nucleotides upstream of a given coding sequence (e.g., upstream of the coding sequence for genes). One of skill in the art will appreciate that a promoter location may vary outside these parameters for some genes, and also that some genes may comprise more than one promoter (e.g., multiple tissue specific promoters).

As used herein, “kits” are understood to contain at least one non-standard laboratory reagent for use in the methods of the invention in appropriate packaging, optionally containing instructions for use. The kit can further include any other components required to practice the method of the invention, as dry powders, concentrated solutions, or ready to use solutions. In some embodiments, the kit comprises one or more containers that contain reagents for use in the methods of the invention; such containers can be boxes, ampules, bottles, vials, tubes, bags, pouches, blister-packs, or other suitable container forms known in the art. Such containers can be made of plastic, glass, laminated paper, metal foil, or other materials suitable for holding reagents.

By “marker” is meant any protein or polynucleotide having an alteration in expression level or activity that is associated with a disease or disorder, e.g., myelitis.

By “modulate” is meant alter (increase or decrease). Such alterations are detected by standard art known methods such as those described herein.

The phrase “nucleic acid” as used herein refers to an oligonucleotide, nucleotide, polynucleotide, or to a fragment of any of these, to DNA or RNA of genomic or synthetic origin which may be single-stranded or double-stranded and may represent a sense or antisense strand, peptide nucleic acid (PNA), or to any DNA-like or RNA-like material, natural or synthetic in origin, complementary DNA (cDNA), linear or circular oligomers or polymers of natural and/or modified monomers or linkages, including deoxyribonucleosides, ribonucleosides, substituted and alpha-anomeric forms thereof, locked nucleic acids (LNA), phosphorothioate, methylphosphonate, and the like. Polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means. As will be understood by those of skill in the art, when the nucleic acid is RNA, the deoxynucleotides A, G, C, and T are replaced by ribonucleotides A, G, C, and U, respectively. The degeneracy of the nucleic acid code is well understood. The nucleic acid sequences may be “chimeric,” that is, composed of different regions. In the context of this invention “chimeric” compounds are oligonucleotides, which contain two or more chemical regions, for example, DNA region(s), RNA region(s), PNA region(s) etc. Each chemical region is made up of at least one monomer unit, i.e., a nucleotide. These sequences typically comprise at least one region wherein the sequence is modified in order to exhibit one or more desired properties. Further, it is well known that various organisms have preferred codon usage, etc. Determination of a nucleic acid sequence to encode any polypeptide is well within the ability of those of skill in the art. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

“Primer set” means a set of oligonucleotides that may be used, for example, for PCR. A primer set would consist of at least 2, 4, 6, 8, 10, 12, 14, 16, 18, 20, 30, 40, 50, 60, 80, 100, 200, 250, 300, 400, 500, 600, or more primers.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

By “reduces” is meant a negative alteration of at least 10%, 25%, 50%, 75%, or 100%.

By “reference” is meant a standard or control condition.

A “reference sequence” is a defined sequence used as a basis for sequence comparison or a gene expression comparison. A reference sequence may be a subset of or the entirety of a specified sequence; for example, a segment of a full-length cDNA or gene sequence, or the complete cDNA or gene sequence. For nucleic acids, the length of the reference nucleic acid sequence will generally be at least about 40 nucleotides, preferably at least about 60 nucleotides, more preferably at least about 75 nucleotides, and even more preferably about 100 nucleotides or about 300 or about 500 nucleotides or any integer thereabout or there between.

The term “variant,” when used in the context of a polynucleotide sequence, may encompass a polynucleotide sequence related to a wild type gene. This definition may also include, for example, “allelic,” “splice,” “species,” or “polymorphic” variants. A splice variant may have significant identity to a reference molecule, but will generally have a greater or lesser number of polynucleotides due to alternate splicing of exons during mRNA processing. The corresponding polypeptide may possess additional functional domains or an absence of domains. Species variants are polynucleotide sequences that vary from one species to another. Of particular utility in the invention are variants of wild type gene products. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence. The resulting polypeptides generally will have significant amino acid identity relative to each other. A polymorphic variant is a variation in the polynucleotide sequence of a particular gene between individuals of a given species. Polymorphic variants also may encompass “single nucleotide polymorphisms” (SNPs) or single base mutations in which the polynucleotide sequence varies by one base. The presence of SNPs may be indicative of, for example, a certain population with a propensity for a disease state, that is susceptibility versus resistance. Derivative polynucleotides include nucleic acids subjected to chemical modification, for example, replacement of hydrogen by an alkyl, acyl, or amino group. Derivatives, e.g. derivative oligonucleotides, may comprise non-naturally-occurring portions, such as altered sugar moieties or inter-sugar linkages. Exemplary among these are phosphorothioate and other sulfur containing species which are known in the art. Derivative nucleic acids may also contain labels, including radionucleotides, enzymes, fluorescent agents, chemiluminescent agents, chromogenic agents, substrates, cofactors, inhibitors, magnetic particles, and the like.

By “VPS37a nucleic acid molecule” is meant a polynucleotide encoding a VPS37a polypeptide. An exemplary VPS37a nucleic acid molecule is provided at NCBI Accession No. NM_152415.2 (SEQ ID NO: 3), and reproduced below:

>NM_152415.2
(SEQ ID NO: 3)
gcagagggggcggagagcgcccccgggggcggggcacgcaagtgacggcg
gcgcgggtggtggagcgctgggcggccaggctccctggctggccggtttg
ggcgtctgggccgtgaaggtgggacctcctgttccgggccgcaagtttcc
ctctccagccgcccgccgttcgtagcatgtcccccagaactcggggagcg
caggcaggacaggcttagagaagacgcggtccccagcgcttgggccacgg
acgtcccaccccgctcctctgtcgctggagaaccgccgggccgagccact
gggagaagcaggccagagccttccagggcctccggcccgtggacccgagg
aggatgagctggctttttcccctgaccaagagcgcctcctcctccgcggc
tgggtcccccggtggcctcaccagcctccagcagcagaagcagcgcctga
tcgagtccctccggaactcacactccagtatagccgaaatacagaaagat
gtggaatacagattgccattcaccataaacaacctgacaattaacattaa
tatattgcttcctccacagtttcctcaagaaaaaccagtgatcagtgttt
atccaccaatacgacatcacttaatggataaacaaggagtgtatgttacc
tctccattagtaaacaattttacaatgcactcagatcttggaaaaattat
tcagagtctgttggatgagttttggaagaatcctccagttttagctccta
cttcaacagcatttccttatctatacagtaacccaagtgggatgtctcct
tatgcttctcagggttttccatttcttcctccatatcctccacaagaagc
aaacaggagtatcacttctttatctgttgctgacactgtttcttcttcaa
caacaagtcataccacagccaagcctgccgctccttcatttggtgtcctt
tcaaatctgccattacccattcccacagtggatgcttcaataccgacaag
ccaaaatggttttgggtacaagatgccagatgtccctgatgcatttccag
aactctcagaactaaatgtgtcacaactcacagatatgaatgaacaagag
gaggtattactagaacagtttctgactttgcctcaactaaaacaaattat
taccgacaaagatgacttagtaaaaagtattgaggaactagcaagaaaaa
atctccttttggagcccagcttggaagccaaaagacaaactgttttagat
aagtatgaattacttacacagatgaagtccactttcgaaaagaagatgca
aaggcagcatgaacttagtgagagctgtagtgcaagtgcccttcaggcaa
gattgaaagtagctgcacatgaagctgaggaagaatctgataatattgca
gaagacttcttggagggaaagatggaaatagatgattttctcagtagctt
cataaaaaagagaacaatttgccactgtagaagagccaaggaagagaaac
ttcagcaggcgatagcaatgcacagccaatttcatgctccactatagatt
ttcctggaaacatgaactgccaagagaggaatgggacacaaaaccaaaca
ctgttttatatttatggtttgcaaactggcatttcatcagtggctaaatt
cacagatatcctatatagattgtatacagaactgagactgattttgtacc
gattagaatgattgctatgatctttgagaaatttttctgcactatttgca
ctgaaatgtttattcattgttgataaattgtatcatatttaagttccact
gctgttcctcttaccttgattaaatgcctatgcatgtacttttagctagt
ttttaatattttataaaacttcatttaaatttgtatttttaacttgaagt
tccatttctttatcaaggatggtatttagatttttttcctcttaaccttt
tttcaaaaactattttcaactgtgaggaaacccttatttttctttctttg
tggataaaactttcaaaagcaatttaagatattcatagtgttaggaaaca
ccaaacctgcctatgtgccatctcacaaaagaaacttttaatacctacaa
taaatcaaaagaataaaccagctgttcttatatattgtttcatttttaaa
actaaagatgcatttaagaagcaatacaagtaaatattttacctaatagg
aaaaaaaaaagttgcctttcatttaaaccattccaacagaaattcttatg
ctaatttaaaacatatatatatctggtaggtttgtggttggataggtttt
ctaaattcctaatgttaaaaacaatctttatgttaatatacactaaatct
atacacaaaaaaagtcagtgaacttttctgacctttactgtgagttacct
tttcctaagaggaaagctatagtaataagtaaaatttaatttttaggcaa
tcctgatttttaatgaatttaattgagtgttcttgtatactacattgagc
agtttgcttctataccgtgtcacaaaattcatgtatttcttgagaagccc
taaaagctcataaaggaaaatgccgtgaactatgtagctcaggcttggta
aggtgccatctaaattacaaaacaaactaatgcataattttgcttaaatt
tcatcccagtatgattgtcttcccaacaccagcatatagtatagattgtc
tgtcttttttatattttttagttcttcctgtacatgtttttggcaataaa
gttataggaagaacaaaattattttgttagaattaaaacatgcttaatat
ttagtctgtttgtggagggcaggtattcacgtggactgagatacaatgtt
ggatacagaaaataactttcattgtcttcctgacactgtgctaaggacat
gctgttaaagcttcaaagtgaccagatgaggaaggaataattaattatta
ctcctgatttgtagataactgaggtaagagtgtttcaaatttatgatagt
cttttgggtattcagaaacctttccttatactgcactggccaccagagct
taattttcccagcagttacagcaatgggagatagaacagtctcaatcttt
tgccaaccatcaggttcctagaaaccaggtaggtgtatcccataacaagg
gaggagcataccacagcccctcatttgattaattcatttgatctatctat
gttattaagtacctactaggaataaggcattgtggaaatactatacaaag
ataaacattgtttagatgcttatctactttccttttcaccagaaaaacag
aaaaaaaagaaacattttcttacagagtaaaaatgttctacataatcaca
tgagtagttcatctcagtgttttttattctttaaagttgaactatcccag
tttcattctataccattcattggataaccttgttacaacccagtcatgaa
acagagcagtgtgatcagttatctgcatttaacaaatagacaaatcagtt
tacataaaggttatgtatgtcacccacgatgaaaagaatctgcatttgaa
tatgcccgtatgaatgtgggttctgtttttgcaacagagattaagtgacc
attttttctaattttatggctatatattttcttcataaaaattggtcaca
tcggagaagcagtgccacaggaaaaatgaaatgcatgtgaaagtttgtat
tctgattttacaagatgagatagaaatcagaattaaagaggaatacttag
gagttactaggctaatcagtgtacgaatttgtcataggtagagatttaaa
ggttaatatcttaaaatagaagaaaattctaaatcaatcaatcagtgaga
tataaactaaacagacccacttcaaagttgaaagaaatttctaggcataa
attgagactaggaaatttatatcagaatagagggtgcttgacacatatat
atgcttaaattgaaggacagctcagattcatttttaggagaagaaagtaa
actaatgtgctcttaaagaataaaaatttattctatggtttctgtctctg
atcatcaccttccattctataaaaagctcagttactgatttgctgggtca
tggtcaaaattcttacctatttatttcatatcaactttaaaaaataaatt
acttgcattctatatattactaattgggaagtaatatgcctcaaatcagt
tttatactggattattccctatgctttaaaccactgctctcaataaaaca
cttcctgattaatgtttgattattagatattttagtcttgttggggatat
tttagtcttgttgggttagccatgctctgaagaatctgtgaaagtacagt
aaagttttaataagcaataaatgtaaccttttatataaatctcagtgcta
ggttaacttctaataagcagacgaacatgttacataaattataatgtctg
tcttgtaaaaaagttgaggggactaaaagtttatgactctgatatggaag
ttgtcatattaaaaaactacattttaaaacatcaaatatttatactattt
gcttttcaaataaaagcatagtgctgtttggcata

By “VPS37a polypeptide” is meant a polypeptide or fragment thereof having at least about 85% amino acid identity to NCBI Accession No. NP_689628.2 (SEQ ID NO: 4) and having DNA binding activity, as reproduced below:

>NP_689628.2
(SEQ ID NO: 4)
MSWLFPLTKSASSSAAGSPGGLTSLQQQKQRLIESLRNSHSSIAEIQKDV
EYRLPFTINNLTININILLPPQFPQEKPVISVYPPIRHHLMDKQGVYVTS
PLVNNFTMHSDLGKIIQSLLDEFWKNPPVLAPTSTAFPYLYSNPSGMSPY
ASQGFPFLPPYPPQEANRSITSLSVADTVSSSTTSHTTAKPAAPSFGVLS
NLPLPIPTVDASIDTSQNGFGYKMPDVPDAFPELSELSVSQLTDMNEQEE
VLLEQFLTLPQLKQIITDKDDLVKSIEELARKNLLLEPSLEAKRQTVLDK
YELLTQMKSTFEKKMQRQHELSESCSASALQARLKVAAHEAEEESDNIAE
DFLEGKMEIDDFLSSFMEKRTICHCRRAKEEKLQQAIAMHSQFHAPL

The term “sample” as used herein refers to a biological sample obtained for the purpose of evaluation in vitro. Exemplary tissue samples for the methods described herein include tissue samples from blood, sputum, or spinal fluid. With regard to the methods disclosed herein, the sample or patient sample preferably may comprise any body fluid or tissue. In some embodiments, the bodily fluid includes, but is not limited to, blood, plasma, serum, lymph, breast milk, saliva, mucous, semen, vaginal secretions, cellular extracts, inflammatory fluids, cerebrospinal fluid, feces, vitreous humor, or urine obtained from the subject. In some aspects, the sample is a composite panel of at least two of a blood sample, a plasma sample, a serum sample, and a urine sample. In exemplary aspects, the sample comprises blood or a fraction thereof (e.g., plasma, serum, fraction obtained via leukopheresis). Preferred samples are whole blood, serum, plasma, or urine. A sample can also be a partially purified fraction of a tissue or bodily fluid. A sample may also include, for example, saliva, hair follicle, cheek swab, and skin scraping.

A reference sample can be a “normal” sample, from a donor not having the disease or condition fluid, or from a normal tissue in a subject having the disease or condition. A reference sample can also be from an untreated donor or cell culture not treated with an active agent (e.g., no treatment or administration of vehicle only). A reference sample can also be taken at a “zero time point” prior to contacting the cell or subject with the agent or therapeutic intervention to be tested or at the start of a prospective study.

The term “subject” as used herein includes all members of the animal kingdom prone to suffering from the indicated disorder. In some aspects, the subject is a mammal, and in some aspects, the subject is a human. The methods are also applicable to companion animals such as dogs and cats as well as livestock such as cows, horses, sheep, goats, pigs, and other domesticated and wild animals.

A subject “suffering from or suspected of suffering from” a specific disease, condition, or syndrome (e.g., myelitis, idiopathic transverse myelitis, etc.) has a sufficient number of risk factors or presents with a sufficient number or combination of signs or symptoms of the disease, condition, or syndrome such that a competent individual would diagnose or suspect that the subject was suffering from the disease, condition, or syndrome. Methods for identification of subjects suffering from or suspected of suffering from conditions associated with myelitis is within the ability of those in the art. Subjects suffering from, and suspected of suffering from, a specific disease, condition, or syndrome are not necessarily two distinct groups.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, at least 70%, at least 80%, at least 85%, at least 90%, at least 95%, or at least 99% identical at the amino acid level or nucleic acid to the sequence used for comparison.

A “purified” or “biologically pure” gene or protein is sufficiently free of other materials such that any impurities do not materially affect the biological properties of the gene or protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

Similarly, by “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

As used herein, “susceptible to” or “prone to” or “predisposed to” or “at risk of developing” a specific disease or condition refers to an individual who based on genetic, environmental, health, and/or other risk factors is more likely to develop a disease or condition than the general population. An increase in likelihood of developing a disease may be an increase of about 10%, 20%, 50%, 100%, 150%, 200%, or more.

As used herein, the terms “treat,” treating,” “treatment,” and the like refer to reducing or ameliorating a neurological disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to the administration of an agent or composition to a clinically asymptomatic individual who is at risk of developing, susceptible, or predisposed to a particular adverse condition, disorder, or disease, and thus relates to the prevention of the occurrence of symptoms and/or their underlying cause.

Unless specifically stated or obvious from context, as used herein, the term “or” is understood to be inclusive. Unless specifically stated or obvious from context, as used herein, the terms “a,” “an,” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

A “therapeutically effective amount” is an amount sufficient to effect beneficial or desired results, including clinical results. An effective amount can be administered in one or more administrations.

Other features and advantages of the invention will be apparent to those skilled in the art from the following detailed description and claims.

### BRIEF DESCRIPTION OF THE DRAWINGS

FIGS. 1A-1C depict MRI images of the spine (sagittal and axial views) of idiopathic transverse myelitis (ITM) patients homozygous for a VPS37A mutation. FIG. 1A depicts MRI images from Sister 1 with ITM and shows a T2 hyperintense lesion 38 years after onset at T8/T9 (arrows). FIG. 1B depicts MRI images from Sister 2 with ITM and shows an acute T2 hyperintense lesion at T4/T5 (arrows). FIG. 1C depicts an unrelated patient with ITM and shows an acute T2 hyperintense lesion at T3 (arrows).

FIGS. 2A-2D depict a DNA sequence analysis of VPS37A. FIG. 2A depicts that the electropherogram of the patient reveals a nonsynonymous homozygous C-to-A substitution (red arrow) at position 700 bp of VPS37A (c.700C>A, p.Leu234Ile). FIG. 2B depicts that the electropherogram reveals a heterozygous c.700C>A mutation (blue arrow) in a patient without ITM. FIG. 2C depicts a representative electropherogram of VPS37A from normal controls (black arrow). FIG. 2D demonstrates that the p.Leu234 residue is highly conserved and found in evolutionarily distant orthologs from humans to zebrafish. The sequences were derived from GenBank records with the following accession numbers: Homo sapiens (NP_689628.2), Callithrix jacchus (marmoset; XP_008977480.1), Rattus norvegicus (Rat; NP_001020038.1), Mus musculus (house mouse; NP_291038.2), Gallus gallus (chicken; XP_420687.1), Xenopus laevis (African clawed frog; XP_018105271.1), Danio rerio (zebrafish; NP_956284.1).

### DETAILED DESCRIPTION

The present disclosure provides compositions and methods for detecting myelitis. In particular, the present disclosure provides methods of identifying mutations in VPS37a indicative of monophasic acute transverse myelitis.

### Transverse Myelitis

Idiopathic transverse myelitis (ITM) is a monophasic autoimmune attack on the spinal cord that leads to weakness, numbness, and bowel/bladder dysfunction. The incidence is approximately 1 per million per year (0.1/100,000) with a prevalence of approximately 7,500 Americans living with disability from their ITM today. There is a bimodal age distribution with a peak in the teenage years and one in adulthood, with men and women being equally affected. Treatment involves acute suppression of the immune system at the time of the attack but neurologic disability persists in greater than two thirds of cases. The etiology of ITM is presumed to be an immune mediated attack 2-3 weeks following a systemic infection possibly due to molecular mimicry.

### Nucleic Acids

As described herein, any nucleic acid specimen, in purified or nonpurified form, can be utilized as the starting nucleic acid or acids, provided it contains, or is suspected of containing, the specific nucleic acid sequence containing the target locus.

When complementary strands of nucleic acid or acids are separated, regardless of whether the nucleic acid was originally double or single stranded, the separated strands are ready to be used as a template for the synthesis of additional nucleic acid strands. This synthesis is performed under conditions allowing hybridization of primers to templates to occur. Generally synthesis occurs in a buffered aqueous solution, preferably at a pH of 7-9, most preferably about 8. Preferably, a molar excess (for genomic nucleic acid, usually about 10⁸:1 primer:template) of the two oligonucleotide primers is added to the buffer containing the separated template strands. It is understood, however, that the amount of complementary strand may not be known if the process of the invention is used for diagnostic applications, so that the amount of primer relative to the amount of complementary strand cannot be determined with certainty. As a practical matter, however, the amount of primer added will generally be in molar excess over the amount of complementary strand (template) when the sequence to be amplified is contained in a mixture of complicated long-chain nucleic acid strands. A large molar excess is preferred to improve the efficiency of the process.

The deoxyribonucleoside triphosphates dATP, dCTP, dGTP, and dTTP are added to the synthesis mixture, either separately or together with the primers, in adequate amounts and the resulting solution is heated to about 90° C.-100° C. from about 1 to 10 minutes, preferably from 1 to 4 minutes. After this heating period, the solution is allowed to cool to room temperature, which is preferable for the primer hybridization. To the cooled mixture is added an appropriate agent for effecting the primer extension reaction (called herein “agent for polymerization”), and the reaction is allowed to occur under conditions known in the art. The agent for polymerization may also be added together with the other reagents if it is heat stable. This synthesis (or amplification) reaction may occur at room temperature up to a temperature above which the agent for polymerization no longer functions. Thus, for example, if DNA polymerase is used as the agent, the temperature is generally no greater than about 40° C. Most conveniently the reaction occurs at room temperature.

In certain preferred embodiments, the agent for polymerization may be any compound or system which will function to accomplish the synthesis of primer extension products, including enzymes. Suitable enzymes for this purpose include, for example, E. coli DNA polymerase I, Klenow fragment of E. coli DNA polymerase I, T4 DNA polymerase, other available DNA polymerases, polymerase muteins, reverse transcriptase, and other enzymes, including heat-stable enzymes (i.e., those enzymes which perform primer extension after being subjected to temperatures sufficiently elevated to cause denaturation). Suitable enzymes will facilitate combination of the nucleotides in the proper manner to form the primer extension products which are complementary to each locus nucleic acid strand. Generally, the synthesis will be initiated at the 3′ end of each primer and proceed in the 5′ direction along the template strand, until synthesis terminates, producing molecules of different lengths. There may be agents for polymerization, however, which initiate synthesis at the 5′ end and proceed in the other direction, using the same process as described above.

In nucleic acid hybridization reactions, the conditions used to achieve a particular level of stringency will vary, depending on the nature of the nucleic acids being hybridized. For example, the length, degree of complementarity, nucleotide sequence composition (e.g., GC v. AT content), and nucleic acid type (e.g., RNA v. DNA) of the hybridizing regions of the nucleic acids can be considered in selecting hybridization conditions. An additional consideration is whether one of the nucleic acids is immobilized, for example, on a filter.

### Polymerase Chain Reaction (PCR)-Based Amplification Techniques

Preferably, the method of amplifying is by PCR, as described herein and as is commonly used by those of ordinary skill in the art. Alternative methods of amplification have been described and can also be employed as long as the loci amplified by PCR using the primers of the invention are similarly amplified by the alternative means. PCR based detection may include multiplex amplification of a plurality of markers simultaneously. For example, it is well known in the art to select PCR primers to generate PCR products that do not overlap in size and can be analyzed simultaneously. Alternatively, it is possible to amplify different markers with primers that are differentially labeled and thus can each be differentially detected. Other techniques are known in the art to allow multiplex analyses of a plurality of markers.

Several methods have been developed to facilitate analysis of single nucleotide polymorphisms in genomic DNA or cellular RNA. In one embodiment, the single base polymorphism can be detected by using a specialized exonuclease-resistant nucleotide, as disclosed, e.g., U.S. Pat. No. 4,656,127. According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

A primer may be employed that is complementary to allelic sequences immediately 3′ to a polymorphic site. The method determines the identity of the nucleotide of that site using labeled dideoxynucleotide derivatives, which, if complementary to the nucleotide of the polymorphic site, will become incorporated onto the terminus of the primer. Mixtures of labeled terminators and a primer that is complementary to the sequence 3′ to a polymorphic site may be used. The labeled terminator that is incorporated is thus determined by, and complementary to, the nucleotide present in the polymorphic site of the target molecule being evaluated.

The amplified products are preferably identified by sequencing. Sequences amplified by the methods of the invention can be further evaluated, detected, cloned, sequenced, and the like, either in solution or after binding to a solid support, by any method usually applied to the detection of a specific DNA sequence such as PCR, oligomer restriction, allele-specific oligonucleotide (ASO) probe analysis, oligonucleotide ligation assays (OLAs), and the like.

In some embodiments, sequencing techniques such as quantitative PCR (qPCR; or real-time PCR) are performed. Quantitative PCR monitors the amplification of a targeted DNA molecule during the PCR (i.e., in real-time), and not at its end, as in conventional PCR. Real-time PCR can be used quantitatively (quantitative real-time PCR), and semi-quantitatively, i.e. above/below a certain amount of DNA molecules (semi quantitative real-time PCR). Two common methods for the detection of PCR products in real-time PCR are: (1) non-specific fluorescent dyes that intercalate with any double-stranded DNA, and (2) sequence-specific DNA probes consisting of oligonucleotides that are labelled with a fluorescent reporter which permits detection only after hybridization of the probe with its complementary sequence.

Real time chemistry allows for the detection of PCR amplification during the early phases of the reactions, and makes quantitation of DNA and RNA easier and more precise. A few variations of the real-time PCR are known. They include the TAQMAN system and MOLECULAR BEACON system which have separate probes labeled with a fluorophore and a fluorescence quencher. In the SCORPION system the labeled probe in the form of a hairpin structure is linked to the primer.

The conditions generally required for PCR include temperature, salt, cation, pH1 and related conditions needed for efficient copying of the master-cut fragment. PCR conditions include repeated cycles of heat denaturation (i.e. heating to at least about 95° C.) and incubation at a temperature permitting primer: adaptor hybridization and copying of the master-cut DNA fragment by the amplification enzyme. Heat stable amplification enzymes like the pwo, Thermus aquaticus or Thermococcus litoralis DNA polymerases which eliminate the need to add enzyme after each denaturation cycle, are commercially available. The salt, cation, pH and related factors needed for enzymatic amplification activity are available from commercial manufacturers of amplification enzymes.

As provided herein an amplification enzyme is any enzyme which can be used for in vitro nucleic acid amplification, e.g. by the above-described procedures. Such amplification enzymes include pwo, Escherichia coli DNA polymerase I, Klenow fragment of E. coli polymerase I, T4 DNA polymerase, T7 DNA polymerase, Thermus aquaticus (Taq) DNA polymerase, Thermococcus litoralis DNA polymerase, SP6 RNA polymerase, T7 RNA polymerase, T3 RNA polymerase, T4 polynucleotide kinase, Avian Myeloblastosis Virus reverse transcriptase, Moloney Murine Leukemia Virus reverse transcriptase, T4 DNA ligase, E. coli DNA ligase or Q.beta. replicase. Preferred amplification enzymes are the pwo and Taq polymerases. The pwo enzyme is especially preferred because of its fidelity in replicating DNA.

### Primers

The term “primer” as used herein refers to a sequence comprising two or more deoxyribonucleotides or ribonucleotides, preferably more than three, and most preferably more than 8, which sequence is capable of initiating synthesis of a primer extension product, which is substantially complementary to a polymorphic locus strand. Environmental conditions conducive to synthesis include the presence of nucleoside triphosphates and an agent for polymerization, such as DNA polymerase, and a suitable temperature and pH. The primer is preferably single stranded for maximum efficiency in amplification, but may be double stranded. If double stranded, the primer is first treated to separate its strands before being used to prepare extension products. Preferably, the primer is an oligodeoxy ribonucleotide. The primer must be sufficiently long to prime the synthesis of extension products in the presence of the inducing agent for polymerization. The exact length of primer will depend on many factors, including temperature, buffer, and nucleotide composition. The oligonucleotide primer typically contains 12-20 or more nucleotides, although it may contain fewer nucleotides.

Primers of the invention are designed to be “substantially” complementary to each strand of the oligonucleotide to be amplified and include the appropriate nucleotides as discussed above. This means that the primers must be sufficiently complementary to hybridize with their respective strands under conditions that allow the agent for polymerization to perform. In other words, the primers should have sufficient complementarity with a 5′ and 3′ oligonucleotide to hybridize therewith and permit amplification of a nucleic acid sequence.

Primers of the invention are employed in the amplification process, which is an enzymatic chain reaction that produces exponentially increasing quantities of target locus relative to the number of reaction steps involved (e.g., polymerase chain reaction or PCR). Typically, one primer is complementary to the negative (−) strand of the locus (antisense primer) and the other is complementary to the positive (+) strand (sense primer). Annealing the primers to denatured nucleic acid followed by extension with an enzyme, such as the large fragment of DNA Polymerase I (Klenow) and nucleotides, results in newly synthesized + and − strands containing the target locus sequence. Because these newly synthesized sequences are also templates, repeated cycles of denaturing, primer annealing, and extension results in exponential production of the region (i.e., the target locus sequence) defined by the primer. The product of the chain reaction is a discrete nucleic acid duplex with termini corresponding to the ends of the specific primers employed.

The oligonucleotide primers used in invention methods may be prepared using any suitable method, such as conventional phosphotriester and phosphodiester methods or automated embodiments thereof. In one such automated embodiment, diethylphos-phoramidites are used as starting materials and may be synthesized as described by Beaucage, et al. (Tetrahedron Letters, 22:1859-1862, 1981). One method for synthesizing oligonucleotides on a modified solid support is described in U.S. Pat. No. 4,458,066.

The primers of the invention embrace oligonucleotides of sufficient length and appropriate sequence so as to provide specific initiation of polymerization on a significant number of nucleic acids in the polymorphic locus. Where the nucleic acid sequence of interest contains two strands, it is necessary to separate the strands of the nucleic acid before it can be used as a template for the amplification process. Strand separation can be effected either as a separate step or simultaneously with the synthesis of the primer extension products. This strand separation can be accomplished using various suitable denaturing conditions, including physical, chemical, or enzymatic means, the word “denaturing” includes all such means. One physical method of separating nucleic acid strands involves heating the nucleic acid until it is denatured. Typical heat denaturation may involve temperatures ranging from about 80° to 105° C. for times ranging from about 1 to 10 minutes. Strand separation may also be induced by an enzyme from the class of enzymes known as helicases or by the enzyme RecA, which has helicase activity, and in the presence of riboATP, is known to denature DNA. The reaction conditions suitable for strand separation of nucleic acids with helicases are described by Kuhn Hoffmann-Berling (CSH-Quantitative Biology, 43:63, 1978) and techniques for using RecA are reviewed in C. Radding (Ann. Rev. Genetics, 16:405-437, 1982).

### Primer Tags (Molecular Beacons, Fluorophors, and Other Tags)

In some embodiments, primers of the present invention are labeled with detectable tags or moieties. The primer of interest may be detectably labeled, for example, with a radioisotope, a fluorescent compound, a bioluminescent compound, a chemiluminescent compound, a molecular beacon, a metal chelator, or an enzyme. Those of ordinary skill in the art will know of other suitable labels for binding to the primer, or will be able to ascertain such, using routine experimentation.

In some embodiments, molecular beacons are used as labels for primers. Molecular beacons (or molecular beacon probes) are oligonucleotide hybridization probes that may indicate the presence of specific nucleic acids in homogenous solutions. They are hairpin shaped molecules with an internally quenched fluorophore whose fluorescence is activated upon binding to a target nucleic acid sequence. Molecular beacons may be useful in situations where it is either not possible or desirable to isolate the probe-target hybrids from an excess of the hybridization probes.

A typical molecular beacon probe is 25 nucleotides long. In some embodiments, the middle 15 nucleotides are complementary to the target DNA or RNA and do not base pair with one another, while the five nucleotides at each terminus are complementary to each other rather than to the target DNA. A typical molecular beacon structure can be divided in 4 parts: 1) loop, an 18-30 base pair region of the molecular beacon that is complementary to the target sequence; 2) stem formed by the attachment to both termini of the loop of two short (5 to 7 nucleotide residues) oligonucleotides that are complementary to each other; 3) 5′ fluorophore at the 5′ end of the molecular beacon, a fluorescent dye is covalently attached; 4) 3′ quencher (non fluorescent) dye that is covalently attached to the 3′ end of the molecular beacon. When the beacon is in closed loop shape, the quencher resides in proximity to the fluorophore, which results in quenching the fluorescent emission of the latter.

If the nucleic acid to be detected is complementary to the strand in the loop, the event of hybridization occurs. The duplex formed between the nucleic acid and the loop is more stable than that of the stem because the former duplex involves more base pairs. This causes the separation of the stem and hence of the fluorophore and the quencher. Once the fluorophore is no longer next to the quencher, illumination of the hybrid with light results in the fluorescent emission. The presence of the emission indicates that the event of hybridization has occurred and hence the target nucleic acid sequence is present in the test sample.

This invention is further illustrated by the following examples, which should not be construed as limiting. The contents of all references, patents, and published patent applications cited throughout this application, as well as the figures, are incorporated herein by reference.

### EXAMPLES

### Example 1: Detecting Expression of VPS37A Mutations in Idiopathic Transverse Myelitis

ITM is conventionally viewed as a sporadic disease, with no strong familial risk factors and no recognized genetic contribution to risk. Recently, two sisters who are both affected by ITM, one presenting originally in 1967 at age 15 and one presenting in 2010 at age 50 were studied. This unusual occurrence prompted a search for a genetic basis for ITM. Exome sequencing of these sisters revealed that they share homozygosity for a likely deleterious, non-synonymous mutation in exon 6 of VPS37A (c.700C>A, p.Leu234Ile). VPS37A encodes a protein component of the endosomal sorting complex required for transport I protein complex involved in the sorting of ubiquitinated proteins into internal vesicles. The frequency of a heterozygous mutated allele is 0.2/1000-1.5/1000 in the general population, which calculates to an expected homozygous prevalence of 0.004-2.25/100,000.

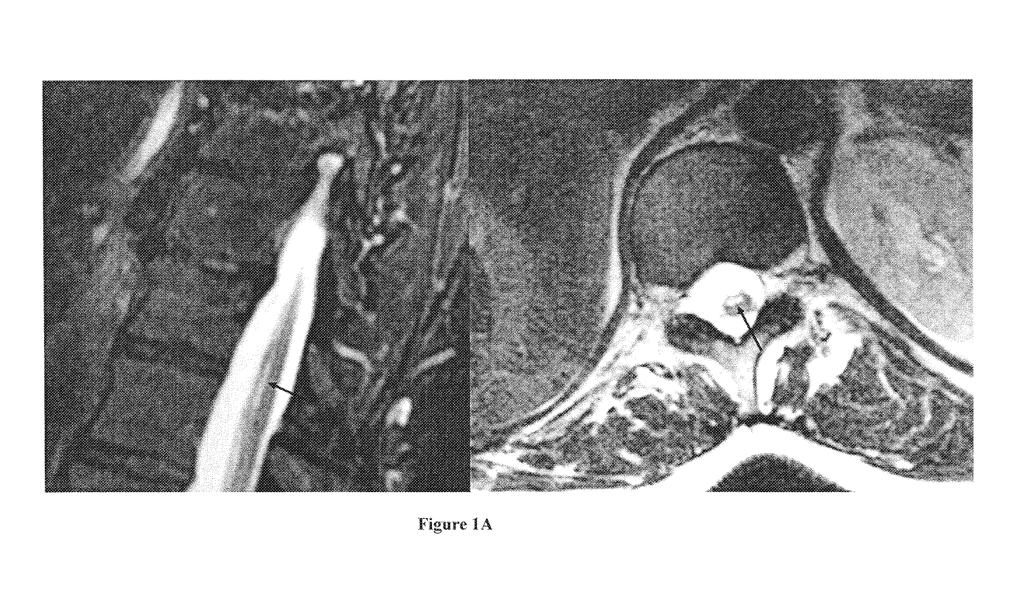

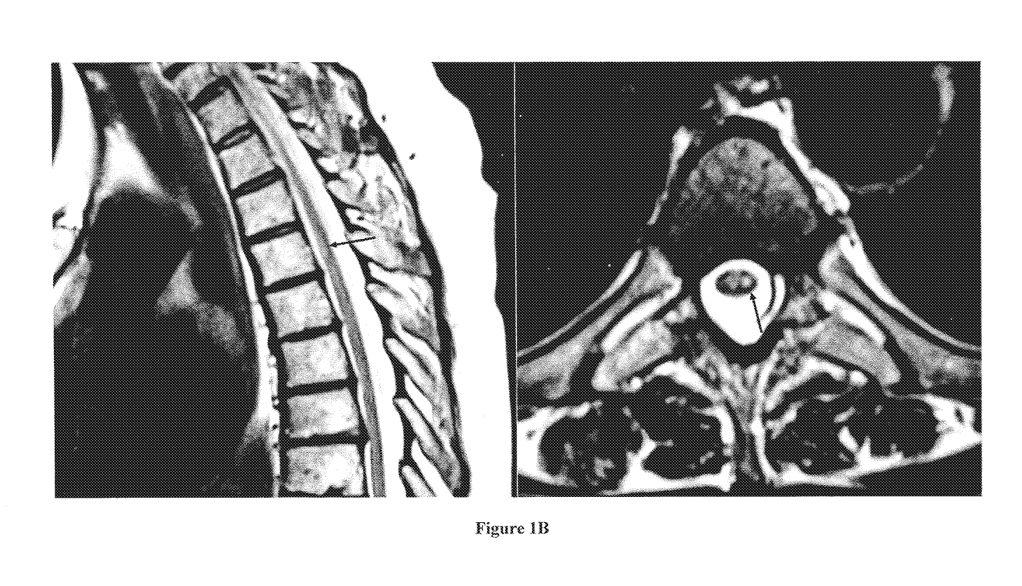

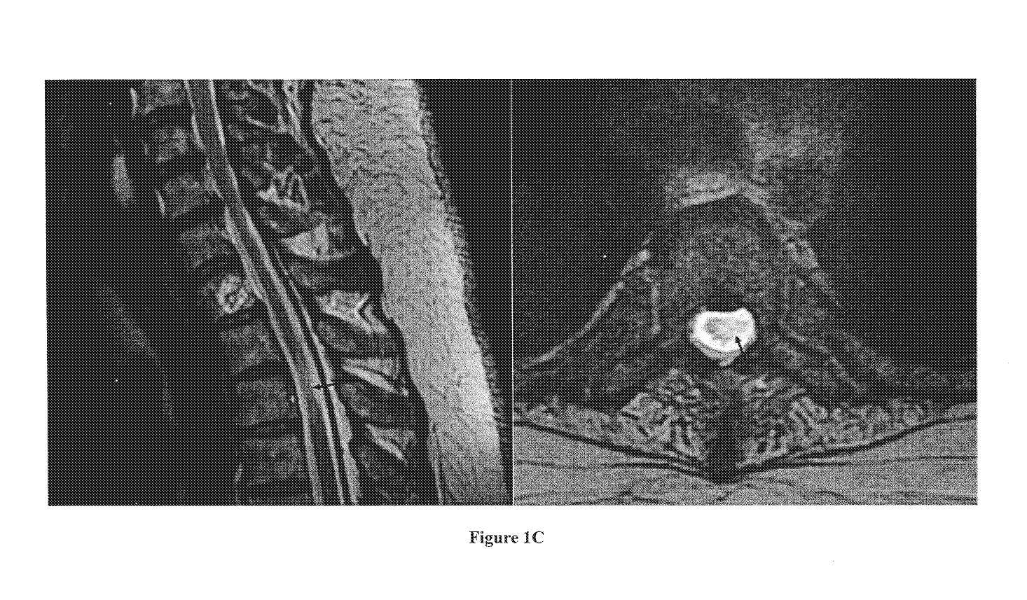

Proband Cases. Sister 1 is currently 64 years old but woke up at age 15 years with abrupt lower extremity weakness and sensory loss. She was treated with steroids and she made a slow and steady recovery over 3 years from a nadir of bedbound to being able to walk without assistance. An MRI performed at age 53 still showed a 12 hyperintense lesion at T8/T9 with no brain lesions (FIG. 1A). No additional neurological events have occurred in the 49 years since her ITM. Sister 2 is currently 59 years old and was 51 when she developed acute onset of low back pain, followed by sensory loss in both legs, mild leg weakness and urinary incontinence. Exam revealed normal strength and a sensory level at T10. MRI of her spinal cord revealed a non-enhancing T2 hyperintense lesion at T4/T5 (FIG. 1B). She was treated with a 1-month course of oral steroids and made a near full recovery except for a persistent mild leg weakness and sensory level on exam. Spinal fluid analysis four months later revealed no pleocytosis nor oligoclonal bands and MRI of the brain was unremarkable. No further neurological events occurred in the 8 years since her ITM. The sisters have two brothers who are both healthy. There is no other known family history of ITM.

### Example 2: Exome Sequencing

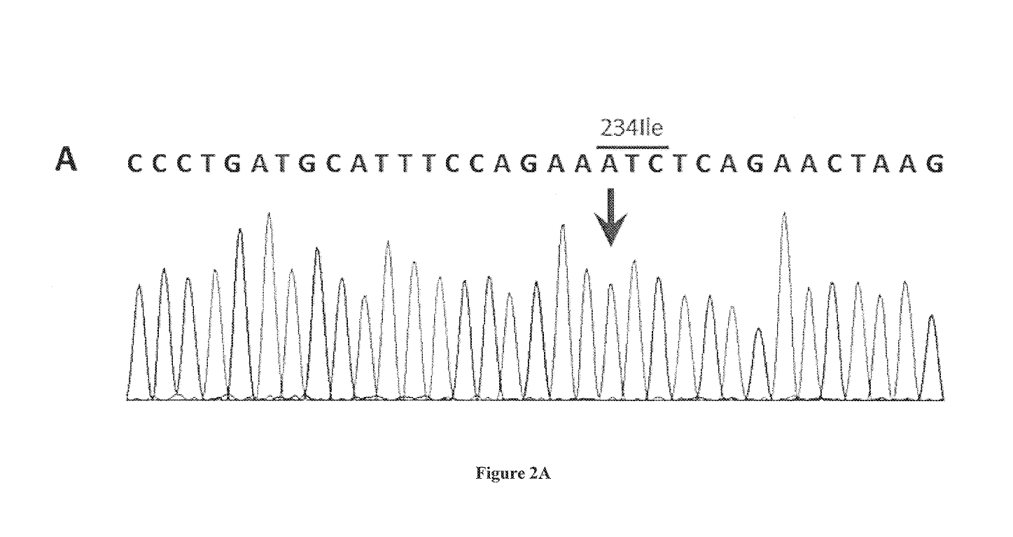

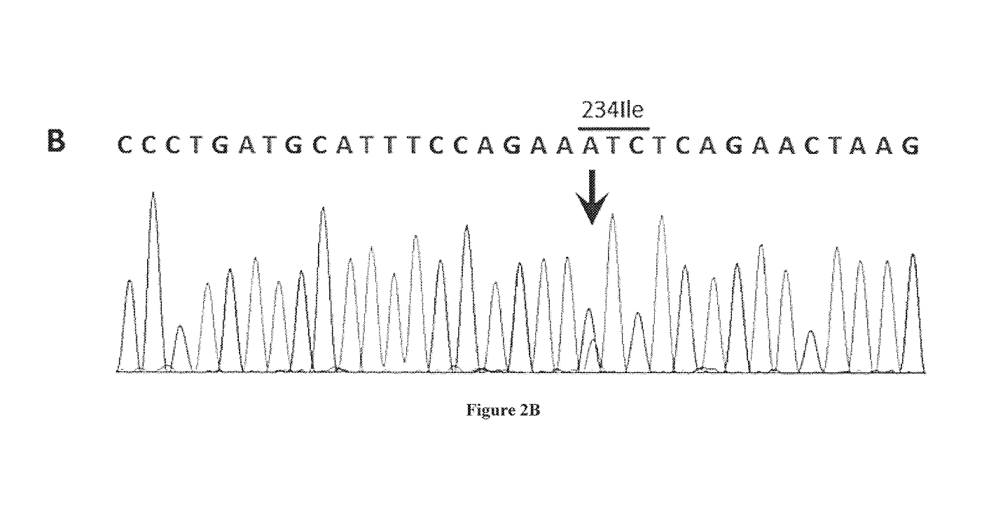

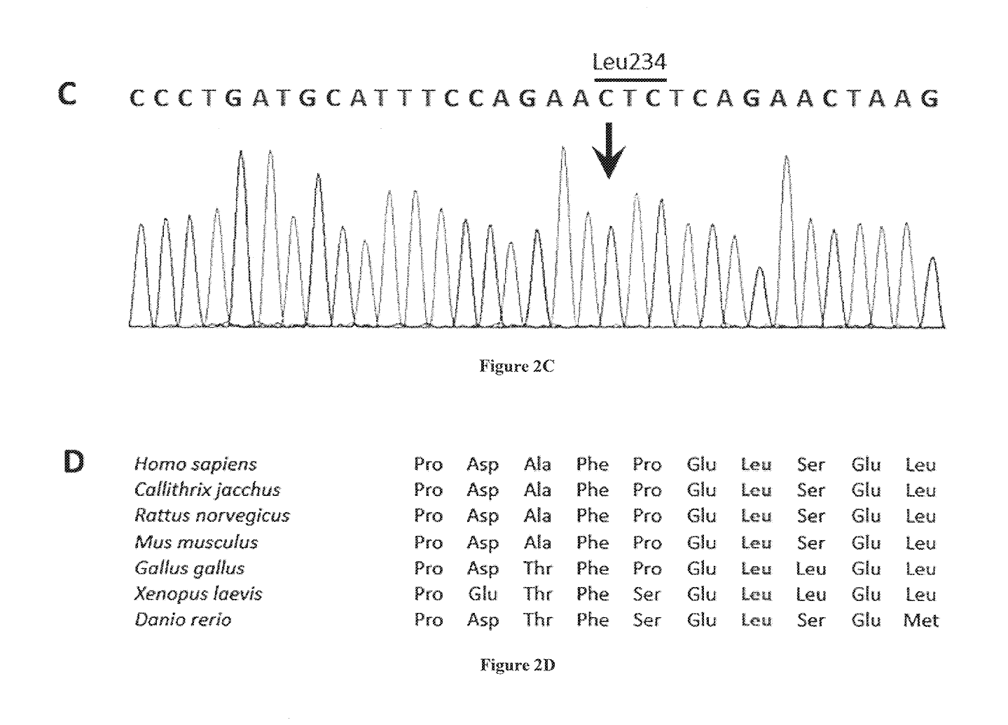

Whole exome sequencing was performed on DNA obtained from the two proband sisters and their two healthy brothers which revealed a non-synonymous, autosomal recessive homozygous mutation in the VPS37A allele (c.700C>A, p.Leu234Ile)(FIGS. 2A-2D). This Leucine to Isoleucine change was expected to be deleterious, and was found randomly as a heterozygous mutation in 4/10,000 individuals who participated in the NHLBI Exome Sequencing Project to 30/10,000 among European descent who participated in the ClinSeq project. The chance of a random homozygous recessive mutation at c.700C>A is therefore calculated to be 1.6- 9/1,000,000. One of the healthy brothers was heterozygous for this mutation. There were no additional homozygous or compound heterozygous candidate variants identified in this analysis.

### Example 3: Screening Among ITM Patients

In addition to the two sisters with ITM, a cohort of 88 subjects with TM and a third ITM patient, unrelated to the sisters, with the same rare autosomal recessive mutation in VPS37A, were screened. These findings strongly implicate VPS37A in the pathogenesis of ITM and for the first time suggest that this autoimmune condition has a genetic component.

An additional 86 ITM samples were screened for this mutation in the VPS37A allele. Another patient with idiopathic TM was identified with the autosomal recessive mutation. This patient was a woman who is currently 55 years old and was 50 years old when acutely developed mid-thoracic back pain and bilateral toe numbness. Physical examination revealed mild left weakness. MRI revealed a T2 hyperintense lesion at T3 and a normal brain MRI (FIG. 1C). Spinal fluid analysis was not performed. She was treated with steroids and made a partial recovery with ongoing back pain and left leg spasms. No further neurological events have occurred since her ITM episode 5 years prior. This woman had no family history of ITM but PCR testing of her sister revealed that her sister carried the heterozygous mutation.

### Example 4: Screening Among Non-ITM Patients

An additional 175 samples from patients with following conditions: multiple sclerosis (n=25), neuromyelitis optica (n=25), healthy controls (n=25), other neurological disease (n=100) were screened for mutations. No homozygous missense mutation in the 700 bp position of VPS37A were identified. However, two heterozygous mutations were detected in a 70 year old man with an inflammatory bony lesion of his skull base with no prior history of myelopathy as well as in the sister of the 3rd ITM with VPS37A c.700C>A mutation described above.

A rare genetic mutation in the VPS37A allele was identified in three patients in two families with a familial form of idiopathic transverse myelitis. The infrequency of this mutation among the general population and the predicted deleterious effect of the Leucine to Isoleucine implicates this genetic mutation in the pathogenesis of familial idiopathic TM. Based on the screening conducted among 88 patients with ITM, 25 with MS, 25 with NMO, 25 healthy controls and 100 with other neurological diseases, it was estimated that up to 3% of idiopathic TM may be associated with this genetic mutation and testing should be strongly considered in patients with a family history of TM.

VPS37A is a component of the endosomal sorting complex required for transport I (ESCRT-1) system which is involved in transporting ubiquitinated cargo to lysosomes for degradation. There are four ESCRT systems comprised of more than 30 proteins that work together in this task and defects in several of these proteins have been associated with neurodegenerative diseases including Alzheimer's, motor neuron disease and hereditary spastic paraplegia (HSP). Two families with an autosomal recessive mutation in VPS37A at a different locus in exon 11, p.K382N, had a marked early onset spastic paraparesis of the arms and legs, intellectual disability, kyphosis, and pectus carinatum. Despite involvement of the same gene, VPS37A, these family members with the p.K382N mutation had a progressive, non-immune-mediated, degenerative course with normal MRI findings. In contrast, ITM patients with the p.Leu234Ile mutation in exon 6 of VPS37A had a single, immune-mediated attack of the spinal cord confirmed by a focal MRI lesion in the spinal cord, followed a stable or improved clinical course. The mechanism that underlies how a mutation in VPS37A leads to a monophasic immune-mediated attack confined to the spinal cord is unknown, however, there is a growing list of autoimmune conditions associated with monogenetic mutation (table 1).

TABLE 1
Autoimmune conditions associated with a monogenetic mutation.

| Autoimmune Condition | Genetic mutation(s) |
|---|---|
| Autoimmune polyglandular | autoimmune regulator (AIRE) |
| syndrome type 1 (APS1) | |
| Immune dysregulation, | forkhead box protein 3 (FoxP3) |
| polyendocrinopathy, enteropathy, | |
| X-linked (IPEX) syndrome | |
| Aicardi - Goutières | TREX1, RNASE H2, SAMHD1, |
| syndrome | ADAR1 |
| C1q deficiency | C1QA, C1QB or C1QC |
| Spondyloenchondrodysplasia with | ACP5 |
| immune dysregulation (SPENCDI) | |
| Type 1 diabetes and | AIRE, FOXP3, IL2RA, ITCH, |
| autoimmune thyroiditis | LRBA, STAT1, STAT3 |

This study was limited to this single mutation in exon 6 of VPS37A. Other TM patients will be screened for additional mutations in the other 11 exons of VPS37A, or in other genes in the ESCRT system. Mouse models of the p.Leu234Ile mutation in development will shed light on the mechanism of how a genetic mutation can lead to vulnerability to a monophasic immune-mediated process in the spinal cord.

### Example 5: Conclusions

A genetic risk for development of idiopathic transverse myelitis was identified. As described herein, two sisters were identified who both have idiopathic transverse myelitis. Exome sequencing was performed on their DNA samples and compared with the DNA of two healthy siblings. Additional samples from 200 patients with idiopathic transverse myelitis, multiple sclerosis, neuromyelitis optica, other neurological conditions and healthy controls were also sequenced.

The two sisters with idiopathic transverse myelitis both had acute onset of sensory loss in the legs, followed by weakness and bowel/bladder dysfunction. The first sister developed myelitis at age 15 with clinical nadir of complete paralysis. Over the next few years, she recovered her ability to walk without assistance. Recent MRI demonstrated persistent T2 lesion in the lower thoracic cord. The second sister developed myelitis at age 50 with nadir of complete sensory loss from T6 down and paraparesis in the legs, associated with an MRI lesion at T6. She also made a partial recovery with treatment. Both sisters share a non-synonymous homozygous mutation in only one gene, VPS37A (c.700C>A, p.Leu234Ile) in the whole genome analyses. One healthy sibling was heterozygous for this mutation. An additional 261 samples from patients with ITM and neuroimmunological diseases were screened by Sanger sequencing of this portion of VPS37A and identified another idiopathic TM patient with this same rare homozygous mutation. No patients with multiple sclerosis, neuromyelitis optica, other neurological conditions or healthy controls contained a homozygous mutation in VPS37A.

In some embodiments, a mutation in VPS37A predisposes to development of idiopathic transverse myelitis. Further studies will determine the frequency of this mutation in this patient population and how this genetic mutation may contribute to risk of disease.

### Methods and Materials

### Samples.

Genetic samples were either extracted from blood or sputum collected prospectively in a sterile manner, or from blood or spinal fluid pellets collected previously using a DNA isolation kit (Qiagen DNeasy Blood &Tissue Kit, cat #69504).

### Sequencing and Quality Control.

DNA samples were processed according to protocols previously described (Gambin T, Jhangiani S N, Below J E, Campbell I M, Wiszniewski W, Muzny D M, Staples J, Morrison A C, Bainbridge M N, Penney S, McGuire A L, Gibbs R A, Lupski J R, Boerwinkle E. Secondary Finding and Carrier Test Frequencies in a Large Multiethnic Sample. Genome Med, 7, 2015; 54, incorporated herein by reference). Sequencing was performed using Illumina Hi-Seq (San Diego, Calif.) instruments after exome capture with the Baylor Human Genome Sequencing Center VCRome 2.1 (ARIC samples) or CORE (CMG samples) designs. To minimize the influence of differences between the two designs on the results of the comparative analysis, the intersection of the capture designs and excluded variants located outside the regions of overlap were identified. Raw sequence data were post-processed using the Mercury pipeline. The Mercury pipeline performs conversion of raw sequencing data (bcl files) to a fastq format using Casava, mapping of the short reads against a human genome reference sequence (GRCh37) using the Burrows-Wheeler Alignment (BWA), recalibration using GATK, and variant calling using the Atlas2 suite. Finally, Cassandra was used to annotate relevant information about gene names, predicted variant pathogenicity, reference allele frequencies and metadata from external resources, and then to add these to the Variant Call Format (VCF) file.

After initial data processing every sample was evaluated using rigorous QC metrics, including percentage of targets covered at 20× or greater and concordance of single nucleotide polymorphisms (SNPs) calls between exome sequencing and SNP array data. Additionally, each SNP variant call was filtered using the following criteria: low single nucleotide variant (SNV) posterior probability (<0.95), strand-bias of more than 99% variant reads in a single strand direction and total coverage less than tenfold. Moreover, sample level QC for ARIC cohort removed known and blind duplicates, samples with known sex mismatches (indicating sample contamination), samples with missing rate >65% and extreme outliers (e.g., singleton counts). Only samples that passed QC were included in this analysis.

### PCR and Sequencing of VPS37A Allele.

Confirmatory PCR testing of base pair 700 on exon 6 of the VPS37A was performed using primers (forward-aaggcagtgtgagatgtgaaga, and reverse-tcccactaaggcaacaacaa) yielding a 350 bp product that was sequenced by Sanger method using an Applied Biosystems 3730xl DNA Analyzer after cleanup with the Agencourt AMPure XP automated PCR purification system.

### REFERENCES

1. Kaplin, A. I., et al., Diagnosis and management of acute myelopathies. Neurologist, (2005). 11(1): p. 2-18.
2. Krishnan, C., et al., Transverse Myelitis: pathogenesis, diagnosis and treatment. Front Biosci, (2004). 9: p. 1483-99.
3. Frohman, E. M. and D. M. Wingerchuk, Clinical practice. Transverse myelitis. N Engl J Med, (2010). 363(6): p. 564-72.
4. Wunderley, L., et al., The molecular basis for selective assembly of the UBAP1-containing endosome-specific ESCRT-I complex. J Cell Sci, (2014). 127(Pt 3): p. 663-72.
5. Fink, J. K., Hereditary spastic paraplegia: clinico-pathologic features and emerging molecular mechanisms. Acta Neuropathol, (2013). 126(3): p. 307-28.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method of diagnosing myelitis, comprising:
obtaining a sample from a subject;
amplifying a region of a VPS37a gene with a first primer and a second primer, wherein the first primer has the sequence aaggcagtgtgagatgtgaaga (SEQ ID NO: 1), fragments or variants thereof, and the second primer has the sequence tcccactaaggcaacaacaa (SEQ ID NO: 2) fragments or variants thereof;
sequencing the amplified sequence; and
identifying a leucine to isoleucine change at amino acid position 234.

2. The method of claim 1, wherein the subject is human.

3. The method of claim 1, wherein the primer is coupled to a fluorescent probe.

4. A isolated nucleic acid sequence comprising SEQ ID NO: 1, SEQ ID NO: 2, fragments or variants thereof.

5. The isolated nucleic acid sequence of claim 4, wherein SEQ ID NO: 1 and/or SEQ ID NO: 2 are conjugated to a fluorescent probe.

6. A composition comprising a nucleic acid sequence comprising SEQ ID NOS: 1, 2, fragments or variants thereof.

7. The composition of claim 6, wherein the nucleic acid sequences are conjugated to a detectable label.


SEQUENCE LISTING (13 sequences)

SEQ ID NO: 1 - DNA, 22 bp, Artificial Sequence
  Description of Artificial Sequence Synthetic primer
aaggcagtgtgagatgtgaaga

SEQ ID NO: 2 - DNA, 20 bp, Artificial Sequence
  Description of Artificial Sequence Synthetic primer
tcccactaaggcaacaacaa

SEQ ID NO: 3 - DNA, 4585 bp, Homo sapiens
gcagagggggcggagagcgcccccgggggcggggcacgcaagtgacggcggcgcgggtggtggagcgctgggcggccaggctccctggctggccggtttgggcgtctgggccgtgaaggtgggacctcctgttccgggccgcaagtttccctctccagccgcccgccgttcgtagcatgtcccccagaactcggggagcgcaggcaggacaggcttagagaagacgcggtccccagcgcttgggccacggacgtcccaccccgctcctctgtcgctggagaaccgccgggccgagccactgggagaagcaggccagagccttccagggcctccggcccgtggacccgaggaggatgagctggctttttcccctgaccaagagcgcctcctcctccgcggctgggtcccccggtggcctcaccagcctccagcagcagaagcagcgcctgatcgagtccctccggaactcacactccagtatagccgaaatacagaaagatgtggaatacagattgccattcaccataaacaacctgacaattaacattaatatattgcttcctccacagtttcctcaggaaaaaccagtgatcagtgtttatccaccaatacgacatcacttaatggataaacaaggagtgtatgttacctctccattagtaaacaattttacaatgcactcagatcttggaaaaattattcagagtctgttggatgagttttggaagaatcctccagttttagctcctacttcaacagcatttccttatctatacagtaacccaagtgggatgtctccttatgcttctcagggttttccatttcttcctccatatcctccacaagaagcaaacaggagtatcacttctttatctgttgctgacactgtttcttcttcaacaacaagtcataccacagccaagcctgccgctccttcatttggtgtcctttcaaatctgccattacccattcccacagtggatgcttcaataccgacaagccaaaatggttttgggtacaagatgccagatgtccctgatgcatttccagaactctcagaactaagtgtgtcacaactcacagatatgaatgaacaagaggaggtattactagaacagtttctgactttgcctcaactaaaacaaattattaccgacaaagatgacttagtaaaaagtattgaggaactagcaagaaaaaatctccttttggagcccagcttggaagccaaaagacaaactgttttagataagtatgaattacttacacagatgaagtccactttcgaaaagaagatgcaaaggcagcatgaacttagtgagagctgtagtgcaagtgcccttcaggcaagattgaaagtagctgcacatgaagctgaggaagaatctgataatattgcagaagacttcttggagggaaagatggaaatagatgattttctcagtagcttcatggaaaagagaacaatttgccactgtagaagagccaaggaagagaaacttcagcaggcgatagcaatgcacagccaatttcatgctccactatagattttcctggaaacatgaactgccaagagaggaatgggacacaaaaccaaacactgttttatatttatggtttgcaaactggcatttcatcagtggctaaattcacagatatcctatatagattgtatacagaactgagactgattttgtaccgattagaatgattgctatgatctttgagaaatttttctgcactatttgcactgaaatgtttatttattgttgataaattgtatcatatttaagttccactgctgttcctcttaccttgattaaatgcctatgcatgtacttttagctagtttttaatattttataaaacttcatttaaatttgtatttttaacttgaagttccatttctttatcaaggatggtatttagatttttttcctcttaaccttttttcaaaaactattttcaactgtgaggaaacccttatttttctttctttgtggataaaactttcaaaagcaatttaagatattcatagtgttaggaaacaccaaacctgcctatgtgccatctcacaaaagaaacttttaatacctacaataaatcaaaagaataaaccagctgttcttatatattgtttcatttttaaaactaaagatgcatttaagaagcaatacaagtaaatattttacctaataggaaaaaaaaaagttgcctttcatttaaaccattccaacagaaattcttatgctaatttaaaacatatatatatctggtaggtttgtggttggataggttttctaaattcctaatgttaaaaacaatctttatgttaatatacactaaatctatacacaaaaaaagtcagtgaacttttctgacctttactgtgagttaccttttcctaagaggaaagctatagtaataagtaaaatttaatttttaggcaatcctgatttttaatgaatttaattgagtgttcttgtatactacattgagcagtttgcttctataccgtgtcacaaaattcatgtatttcttgagaagccctaaaagctcataaaggaaaatgccgtgaactatgtagctcaggcttggtaaggtgccatctaaattacaaaacaaactaatgcataattttgcttaaatttcatcccagtatgattgtcttcccaacaccagcatatagtatagattgtctgtcttttttatattttttagttcttcctgtacatgtttttggcaataaagttataggaagaacaaaattattttgttagaattaaaacatgcttaatatttagtctgtttgtggagggcaggtattcacgtggactgagatacaatgttggatacagaaaataactttcattgtcttcctgacactgtgctaaggacatgctgttaaagcttcaaagtgaccagatgaggaaggaataattaattattactcctgatttgtagataactgaggtaagagtgtttcaaatttatgatagtcttttgggtattcagaaacctttccttatactgcactggccaccagagcttaattttcccagcagttacagcaatgggagatagaacagtctcaatcttttgccaaccatcaggttcctagaaaccaggtaggtgtatcccataacaagggaggagcataccacagcccctcatttgattaattcatttgatctatctatgttattaagtacctactaggaataaggcattgtggaaatactatacaaagataaacattgtttagatgcttatctactttccttttcaccagaaaaacagaaaaaaaagaaacattttcttacagagtaaaaatgttctacataatcacatgagtagttcatctcagtgttttttattctttaaagttgaactatcccagtttcattctataccattcattggataaccttgttacaacccagtcatgaaacagagcagtgtgatcagttatctgcatttaacaaatagacaaatcagtttacataaaggttatgtatgtcacccacgatgaaaagaatctgcatttgaatatgcccgtatgaatgtgggttctgtttttgcaacagagattaagtgaccattttttctaattttatggctatatattttcttcataaaaattggtcacatcggagaagcagtgccacaggaaaaatgaaatgcatgtgaaagtttgtattctgattttacaagatgagatagaaatcagaattaaagaggaatacttaggagttactaggctaatcagtgtacgaatttgtcataggtagagatttaaaggttaatatcttaaaatagaagaaaattctaaatcaatcaatcagtgagatataaactaaacagacccacttcaaagttgaaagaaatttctaggcataaattgagactaggaaatttatatcagaatagagggtgcttgacacatatatatgcttaaattgaaggacagctcagattcatttttaggagaagaaagtaaactaatgtgctcttaaagaataaaaatttattctatggtttctgtctctgatcatcaccttccattctataaaaagctcagttactgatttgctgggtcatggtcaaaattcttacctatttatttcatatcaactttaaaaaataaattacttgcattctatatattactaattgggaagtaatatgcctcaaatcagttttatactggattattccctatgctttaaaccactgctctcaataaaacacttcctgattaatgtttgattattagatattttagtcttgttggggatattttagtcttgttgggttagccatgctctgaagaatctgtgaaagtacagtaaagttttaataagcaataaatgtaaccttttatataaatctcagtgctaggttaacttctaataagcagacgaacatgttacataaattataatgtctgtcttgtaaaaaagttgaggggactaaaagtttatgactctgatatggaagttgtcatattaaaaaactacattttaaaacatcaaatatttatactatttgcttttcaaataaaagcatagtgctgtttggcata

SEQ ID NO: 4 - protein, 397 aa, Homo sapiens
MSWLFPLTKSASSSAAGSPGGLTSLQQQKQRLIESLRNSHSSIAEIQKDVEYRLPFTINNLTININILLPPQFPQEKPVISVYPPIRHHLMDKQGVYVTSPLVNNFTMHSDLGKIIQSLLDEFWKNPPVLAPTSTAFPYLYSNPSGMSPYASQGFPFLPPYPPQEANRSITSLSVADTVSSSTTSHTTAKPAAPSFGVLSNLPLPIPTVDASIPTSQNGFGYKMPDVPDAFPELSELSVSQLTDMNEQEEVLLEQFLTLPQLKQIITDKDDLVKSIEELARKNLLLEPSLEAKRQTVLDKYELLTQMKSTFEKKMQRQHELSESCSASALQARLKVAAHEAEEESDNIAEDFLEGKMEIDDFLSSFMEKRTICHCRRAKEEKLQQAIAMHSQFHAPL

SEQ ID NO: 5 - DNA, 33 bp, Homo sapiens
ccctgatgcatttccagaaatctcagaactaag

SEQ ID NO: 6 - DNA, 33 bp, Homo sapiens
ccctgatgcatttccagaactctcagaactaag

SEQ ID NO: 7 - protein, 10 aa, Homo sapiens
PDAFPELSEL

SEQ ID NO: 8 - protein, 10 aa, Callithrix jacchus
PDAFPELSEL

SEQ ID NO: 9 - protein, 10 aa, Rattus norvegicus
PDAFPELSEL

SEQ ID NO: 10 - protein, 10 aa, Mus musculus
PDAFPELSEL

SEQ ID NO: 11 - protein, 10 aa, Gallus gallus
PDTFPELLEL

SEQ ID NO: 12 - protein, 10 aa, Xenopus laevis
PETFSELLEL

SEQ ID NO: 13 - protein, 10 aa, Danio rerio
PDTFSELSEM